(19) United States
(12) Patent Application Publication
(10) Pub. No.: US 2024/0033449 A1
(43) Pub. Date: Feb. 1, 2024

(54) METHOD AND SYSTEM FOR DETECTING LEAKS AND/OR VERIFYING ADEQUATE CLOSURE FOLLOWING A MEDICAL PROCEDURE

(71) Applicant: QAELON MEDICAL, STRASBOURG (FR)

(72) Inventor: Eran SHLOMOVITZ, ONTARIO (CA)

(73) Assignee: QAELON MEDICAL, STRASBOURG (FR)

(21) Appl. No.: 18/485,604

(22) Filed: Oct. 12, 2023

Related U.S. Application Data
(63) Continuation-in-part of application No. 16/160,395, filed on Oct. 15, 2018.
Application No. 16/160,395 is a division of application No. 14/668,380, filed on Mar. 25, 2015, now Pat. No. 11,357,932.
(60) Provisional application No. 61/970,188, filed on Mar. 25, 2014.

(51) Int. Cl. A61M 13/00; A61B 1/015; A61B 5/03; A61B 5/145

(52) U.S. Cl. CPC A61M 13/003; A61B 1/015; A61B 5/036; A61B 5/14503; A61B 5/14507; A61M 2202/0283; A61M 2202/0225; A61B 5/0002

(57) ABSTRACT

A hand-held detection device for use in detection of a leakage in an anatomical conduit within a cavity of a body part filled with a gas mixture, the detection device including: an access element configured to penetrate the body part, and a housing, both configured to be held by hand of a user; and a detection module including: a sensor configured to measure at least one gas mixture parameter related to the gas mixture within the cavity; and a sub-computational module arranged in the housing and connected to the at least one sensor, and being configured to compute the gas mixture parameter to provide a gas information, a user interface being configured to communicate the gas information.

### FIELD OF THE INVENTION

The present invention concerns the field of medical procedures, methods, systems and devices, more specifically endoluminal related procedures and systems able to evaluate structural integrity and gas tightness of a lumen, or more generally of a hollow or tubular organ.

The invention is related more precisely to a method and a system for detecting leaks and/or verifying adequate closure following a medical procedure. Most preferably, the invention concerns a method and a system for detecting luminal leaks and/or verifying adequate luminal closure following an endoluminal procedure.

### BACKGROUND OF THE INVENTION

When a medical procedure (mini-invasive or not) is performed on an organ which needs to show under normal circumstances of functioning a fluidic tightness towards its local environment, one must make sure that no discontinuity, nor any passage or opening is present in the organ wall(s) defining the concerned lumen or cavity.

Currently endoluminal therapeutic procedures are gaining in popularity and increasing in complexity. More specifically, therapeutic mini-invasive or endoscopy procedures span nowadays a broad range, from simple polypectomy, fistula, and leak repairs to more advanced procedures, including endoscopic submucosal dissections (ESD), creation of enteric anastamosis, Per Oral Endoscopic Myotomy (POEM), and full thickness resections. These procedures have the potential for tremendous patient and societal benefit, as they tend to be less morbid than other alternative treatments, enabling rapid return to normal activity.

Now, all endoluminal procedures, both diagnostic and therapeutic, are associated with a risk of perforation, at worst, a full thickness, intraperitoneal perforation. Whether created intentionally as part of a transluminal or extra-luminal procedure, or as a result of unintentional complication, these perforations must be securely sealed to prevent post procedural morbidity. Fortunately, numerous endoluminal options are now available for closure of these perforations. Nonetheless, beyond a subjective visual inspection, the endoscopist does not have the ability to objectively test the repair for security of closure.

Physicians, and more particularly the endoscopists, in this situation are faced with two options: the endoscopist may elect to “trust” the repair and observe the patient clinically, or he or she may perform a diagnostic laparoscopy. Neither of these options is ideal. In particular, if the repair was, in fact, adequate, the patient will have been subjected to an unnecessary laparoscopy. Indeed, although laparoscopic leak tests are reliable, they represent an invasive surgical procedure and may negate some of the benefits of a purely endoscopic intervention.

However, on the other hand, significant morbidity may result if a patient is initially observed, only to later present with a leak. This issue continues to hinder the complete reliance on endoscopic closure techniques and the continued adoption of endoluminal therapies is partially hampered by the difficulty in confirming adequate luminal closure.

In view of this situation, some “leak testing” procedures have been proposed in the past, which involve a combination of endoluminal action (e.g. instilling water, colored fluids, or air under pressure) and external observation, whether by direct contact with the organ (US 2005/240093, US 2014/018696, WO 2014/074105), by outside localised detection of gases (US 2014/200437, US 2013/289367) or by sensing the change in physical parameters, such as inside pressure (US2007123781).

Nevertheless, these known leak test methods either require additional invasive surgical procedures, are not sufficiently reliable or accurate and/or need costly and time consuming imaging resources. Given that leak tests have been recognised as an extremely valuable tool, efforts have been made to develop fully endoluminal versions. A technique that makes use of dilute hydrogen gas (H2) in a leak test has been proposed. However, this test may be difficult to implement due to the need for dilute H2, a gas that is not in common clinical use, is highly flammable and is not immediately available in most medical facilities. Moreover, this technique relies on specialized H2 detectors, which also are not in common clinical use.

### SUMMARY OF THE INVENTION

It is an aim of the present invention to overcome the limitations of the existing solutions.

For that purpose and according to one of its aspects, the invention proposes a method for detecting leaks and/or verifying adequate closure following a procedure on a hollow or tubular organ of a subject, wherein a leak test is performed by injecting or insufflating, in the concerned organ, a specific test gas which is not commonly produced or naturally present within the body of the subject, or which is present or produced in a precisely known amount or concentration, and by analysing percutaneously the gas or gas mixture present locally within the body cavity in which said organ is situated, and then verifying the presence, and preferably determining the concentration, of the injected or insufflated test gas in said resident local gas or gas mixture and indicating whether the concerned organ or a lumen defined by the latter is leak-free or not.

In an embodiment of the invention, the leak detection method comprises the step of providing a percutaneous access for detection and possibly quantification of said injected or insufflated test gas, said detection and potential quantification being performed either by sensing or by sampling locally, preferably proximately outside the lumen or organ, the resident gas or gas mixture and, if applicable, transferring the gaseous sample to a gas analysing means.

Advantageously, the percutaneous access is provided by means of an atraumatic access needle, such as a Veress needle, or a trocar, adapted and designed for establishing a fluidic communication line or a passageway between the peritoneal or thoracic cavity of the subject and a gas analysing or sensing means, said access means having possibly been put in place during an earlier procedure. The access needle or trocar may be connected to the remote analysing means via a Luer lock tubing and may have already been placed intraoperatively in an earlier phase for insufflating in, sucking from or managing gases in the body cavity such as CO2 pneumoperitoneum. If not, such a Veress needle or any analogous needle may be put in place with low morbidity and minimal post procedural discomfort.

The detection and possible quantification of the test gas can be performed by transferring at least one sample of the cavity gas mixture to a remotely located detection module or by positioning percutaneously an adapted sensor head at the tip of the access needle or trocar, through the latter, and analysing directly the gas mixture inside the cavity.

In a preferred embodiment of the invention, the test gas is a gas used during anesthesia procedures and having a short elimination half-life, and wherein the used gas analysing means may be part of an anesthesia system.

Most preferably, the injected or insufflated test gas is nitrous oxide (N2O).

Indeed, N2O has several favorable properties making it a good candidate for this application: a 0.46 blood:gas partition coefficient, a minimum alveolar concentration of 104%, and an elimination half-life of 5 minutes. The practical implication of this rapid elimination is that even if used by the anesthesiologist during the procedure, N2O may be rapidly eliminated through the respiratory system and still be utilized subsequently for a leak test according to the invention.

Furthermore, N2O is a safe, low cost inhalational anesthetic that is widely available in the clinical setting. It is distributed in compressed gas tanks which, similar to CO2 tanks, may be easily connected to an endoscopic insufflator. Additionally, virtually every anesthesia machine is equipped with a nitrous oxide detector, which may be easily connected directly to a Veress needle via a simple Luer lock tubing. In various embodiments, the leak test may be performed even when unexpected perforations occur, which is arguably the time in which it is most needed.

Nitrous oxide is a non-flammable, non-irritating gas with diffusion and solubility quotients very similar to CO2. At room temperature, it is relatively chemically inert. N2O has a long history of safe use in anesthesia as a weak inhaled agent, often used in combination with other inhaled anesthetics. However, prior to the present disclosure, N2O has not been used for endoscopic insufflation of a lumen, or for detecting luminal leaks following endoscopic procedures.

Nevertheless, other gases could be good candidates for test gas able to be used in the leak detection method according to the invention such as nitrogen, carbon dioxide or argon.

If there is an insufficient volume of gas inside the cavity to perform good sampling, the method according to the invention may consider having said cavity first inflated with CO2 through a percutaneous access needle, before the test gas is injected or insufflated into the lumen to be checked for gas leaks, the gas or gas mixture within the cavity being then repeatedly sampled and analysed over time.

Advantageously, the concentration variation of test gas can be monitored over time, in particular its time related variation rate, as a parameter indicative of the presence of a leak.

Of course, the same access needle may have been used first for insufflating CO2 into the cavity and then as access or transfer line for the gas analysis.

According to a preferred embodiment, the sampling and analysing step is performed simultaneously with, and/or immediately after, injection or insufflation of the test gas into the concerned lumen, in a synchronised manner.

In relation to an embodiment, the injection of the test gas within the concerned lumen is realised by means of an endoscopic system, preferably of the flexible type. The possible earlier diagnostic, therapeutic or surgical procedure may have taken place in the form of a mini-invasive endoscopic procedure.

Preferably the likelihood of a leak is computed, depending on the case settings, by comparing the measurement data delivered by the analysing means with stored data, for example resulting from previous measurements or inputted by an operator.

Advantageously, the test gas is injected or insufflated into the lumen to be checked, or into a tightly isolated section of the lumen, through a natural channel or orifice, which is sealed during and after the injection or insufflation phase.

Of course, the leak test described hereinbefore may be repeated as needed to re-test the integrity and tightness of the organ or lumen, or possibly the closure after a medical procedure.

According to an other of its aspects, the invention also proposes a system for detecting leaks and/or verifying adequate closure following a medical procedure, on a hollow or tubular organ of a subject, wherein said system is able to perform a leak test and comprises at least an injection module for injecting or insufflating, in the concerned organ, a specific test gas which is not commonly or naturally present or produced within the body of the subject, or present or produced in a precisely known amount or concentration, and a detection module for analysing percutaneously the gas or gas mixture locally present within the body cavity in which said organ is situated and for verifying, in cooperation with a computational module, the presence, and preferably determining the concentration, of the injected or insufflated test gas in said resident local gas or gas mixture and indicating whether or not the concerned organ or a lumen defined by the latter is leak free.

This leak detection system is thus adapted and designed to carry out the aforementioned method. In relation to a preferred embodiment, the system also comprises a user interface for interaction of the user with the system, said interface comprising means for the user to enter information and means to display information to the user, and communicating at least with the computational module.

According to other embodiments, the injection module is adapted to control the injection of the test gas and comprises means to measure or standardise the volume, the concentration and/or the rate of injection of the test gas delivered from a corresponding source, as well as a pressure measurement means to determine the pressure within the injection tubing and/or inside the injected lumen. The detection module may comprise means providing a percutaneous fluidic access, proximate and outside the concerned lumen, to the internal volume of the cavity, wherein the organ with the lumen is located, means for transferring (a) sample(s) of the local gas or gas mixture to outside analysing means able to make a real time presence or concentration measurement of the test gas within the sample(s). The computational module may comprise means to store and to treat data provided by the other modules and by the user interface, means to communicate with and to operatively manage the injection and detection modules, means to retrieve information from the user interface and from a remote data storage and means to send information to a user interface.

In relation to a first constructive embodiment, the injection, detection and computational modules are part of an existing medical system, such as an anesthesia apparatus.

According to a second constructive embodiment, the injection, detection and computational modules, as well as the user interface are all integrated in a same independent housing, said housing also incorporating means to cooperate with a medical setting.

In relation to a third constructive embodiment, the injection, detection and computational modules, as well as the user interface are physically located in at least two separate housings, each housing or each module incorporating means for mutual wireless communication or for mutual communication through a physical link.

Advantageously, the injection module may be incorporated within a separate independent housing and constitutes, is part of or is connected to a handheld instrument, such as a flexible endoscope.

In this case, the test gas may be delivered to an endoscopic insufflator via a flow regulator.

In yet a further embodiment, the injection module and the detection module are located in housings separate from the housing incorporating the computational means, each housing comprising wireless communication means and said injection and detection modules comprising specific user interface means, for control and display.

The user interface, or main user interface, is, if not in the form of a separate unit, normally incorporated within the housing of the computational module.

Nevertheless, it may also consist in a separate device, more precisely a personal portable item belonging to the user or operator, for example a smartphone with a corresponding software application able to communicate at least with the computational module.

### BRIEF DESCRIPTION OF THE DRAWINGS

Embodiments of the present disclosure will be readily understood by the following detailed description in conjunction with the accompanying drawings. To facilitate this description, like reference numerals designate like structural elements. Embodiments are illustrated by way of example and not by way of limitation in the figures of the accompanying drawings, wherein:

FIG. 1 is a schematic representation illustrating a site where the inventive leak testing method is applied;

FIG. 2 is a general synoptic or block diagram of the main components of the system according to a preferred embodiment of the invention;

FIG. 3 is a synoptic view of a first physical construction of the system of FIG. 2, showing a single housing for all components of the system;

FIG. 4 is a functional block diagram of the constructive embodiment of the system of FIG. 3;

FIG. 5 is a synoptic view of a second physical construction of the system of FIG. 2, showing separate housings for the components of the system;

FIG. 6 is a functional block diagram of the injection module which is part of the system shown on FIG. 5;

FIG. 7 is a functional block diagram of the detection module which is part of the system shown on FIG. 5, and,

FIGS. 8 and 9 are alternative functional block diagrams of the computational module which is part of the system shown on FIG. 5, according to two different embodiments of the user interface.

FIG. 10 is a top view in perspective of an embodiment of the detection device configured to be mounted inside a cannula of a trocar, and featuring a user interface on an upper surface of a housing of the detection device.

FIG. 11 is a below view in perspective of the detection device of FIG. 10, showing an access element of the detection device, directly linked to the housing.

FIG. 12 is a cross section of the detection device of FIG. 10, showing components placed inside the housing, and a gas sensor placed inside a lumen of the access element.

FIG. 13 is a schematic view of the detection device of FIG. 10 and a trocar, before assembling.

FIG. 14 is a schematic view of the assembled detection device of FIG. 10 and trocar, illustrating the coupling of an adjustment member within the cannula for holding the detection device with the trocar, a sealing valve of the trocar pressed against the access element thus preventing leakage from the body cavity, and the sealing of an insufflating access hole of the trocar, thanks to a sealing member of the detection device.

FIG. 15 is a schematic view of the use of the detection device of FIG. 10 during a surgery, in which a hollow organ has been surgically treated, and is insufflated in order to check on the presence of a leakage on a suture made during the surgery, with the help of the detection device mounted in a trocar reaching a body cavity surrounding the hollow organ.

FIG. 16 is another schematic view of the use of the detection device during a surgery.

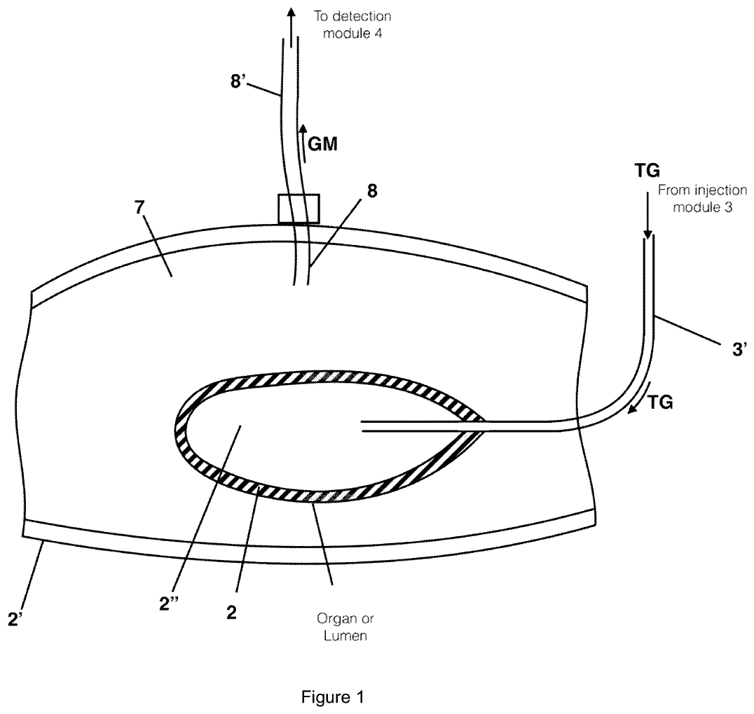

### DETAILED DESCRIPTION OF THE INVENTION

In the following detailed description, reference is made to the accompanying drawings which form a part hereof wherein like numerals designate like parts throughout, and in which is shown by way of illustration embodiments in which the disclosure may be practiced. It is to be understood that other embodiments may be utilized and structural or logical changes may be made without departing from the scope of the present disclosure. Therefore, the following detailed description is not to be taken in a limiting sense, and the scope of embodiments in accordance with the present disclosure is defined by the appended claims and their equivalents.

Various operations may be described as multiple discrete operations in turn, in a manner that may be helpful in understanding embodiments of the present disclosure; however, the order of description should not be construed to imply that these operations are order dependent.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In its most preferred embodiment and application, the present invention is directed towards a method and a system 1 for detecting luminal leaks and/or verifying adequate luminal closure following an endoluminal procedure, within a hollow or tubular organ 2 of a subject 2′.

In other words, the invention encompasses a leak detection method and system for hollow or tubular organs.

The main steps and features of the inventive method have already been introduced hereinbefore and will be understood and explained in more details in relation with the system as described hereinafter, by way of exemplary embodiments.

The inventive system 1 is able and designed to perform a leak test according to the method described before.

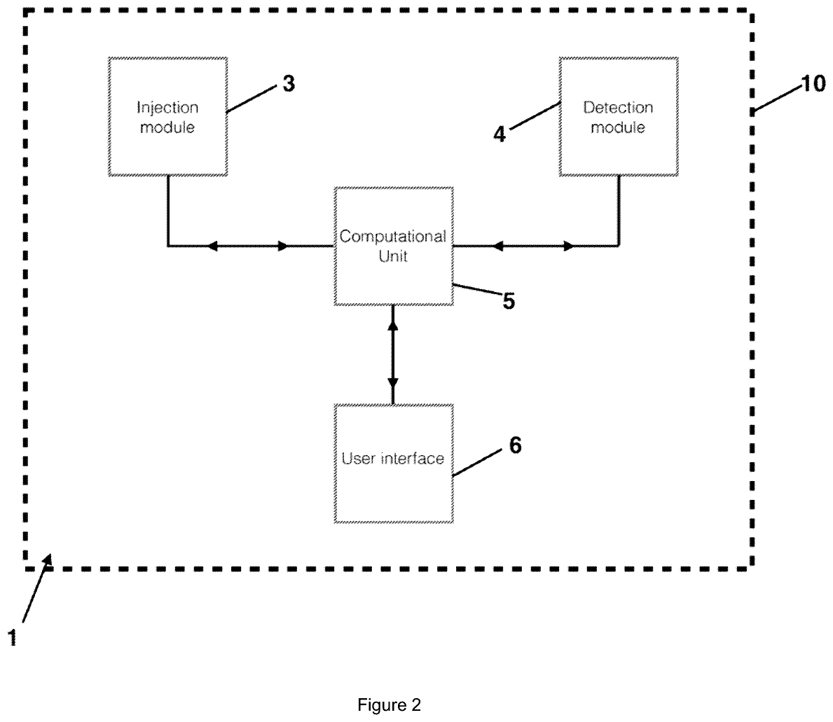

As shown on FIGS. 2, 3 and 5, said system 1 comprises at least an injection module 4 for injecting or insufflating, in the concerned lumen 2″, a specific test gas TG which is not commonly or naturally present or produced within the body of the subject 2′, or present or produced in a precisely known amount or concentration, a detection module 4 for analysing percutaneously the gas or gas mixture GM locally present within the body cavity 7 in which said organ 2′ comprising said lumen 2″ is situated and for verifying, in cooperation with a computational module 5, the presence, and preferably determining the concentration, of the injected or insufflated test gas TG in said resident local gas or gas mixture GM and indicating whether or not the concerned lumen 2″ is leak free.

As also shown on said figures, the system 1 also comprises a user interface 6 for interaction of the user with the system 1, said interface comprising means for the user to enter information and means to display information to the user, and communicating at least with the computational module 5.

As can be seen from the attached schematic drawings and as exposed hereinbefore and after, the invention concerns, on the one hand, an endoluminal leak detection method and, on the other hand, an endoluminal leak detection system, representing two aspects of the same invention.

Although the following specification is more generally directed towards the system 1, the major features inventive method can be easily derived from the various functions performed by the different components 3, 4, 5, 6 of the system 1.

Fundamentally, the system 1 is adapted and designed to control the injection of a test gas TG into a hollow organ 2 to be checked for leaks and to measure if the test gas is leaking in an adjacent space (internal free volume of the body cavity 7 containing the organ 2). The leak detection system 1 computes the likelihood of a leak and this information is then displayed to the user. A computational module 5 is provided to synchronize and control the parameters of the injection process and of the detection process, manage the various components of the system, interpret user input and compute the likelihood of a medically relevant leak in the aforementioned hollow organ 2 and display that information to the user. The computation is based on stored data (possibly retrieved from a remote storage 9), information entered by the user and the gas measurement information.

Thus, the leak detection system I computes the likelihood of a medically relevant leak in the aforementioned hollow organ 2. It generally comprises an injection module 3, a detection module 4, a computational module 5 and a possible user interface 6.

According to various embodiments of the present invention, said leak detection system I is adapted to be used in different clinical settings such as, but not limited to, flexible endoscopy procedures, laparascopic procedures, open surgery procedures, percutaneous procedures or radiologic procedures. Depending on the clinical setting the cooperation and communication between the constitutive means and components of the leak test system I may vary.

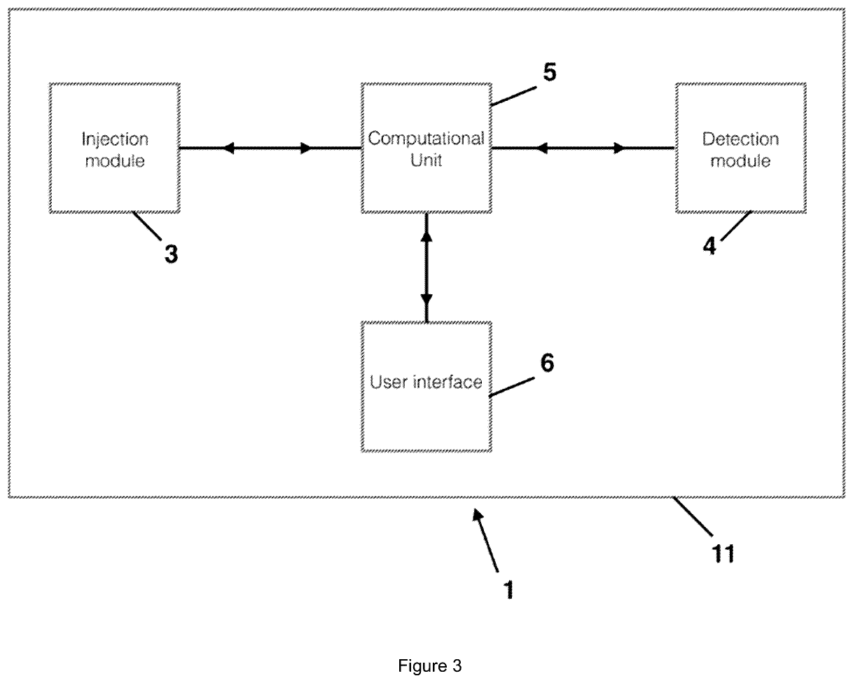

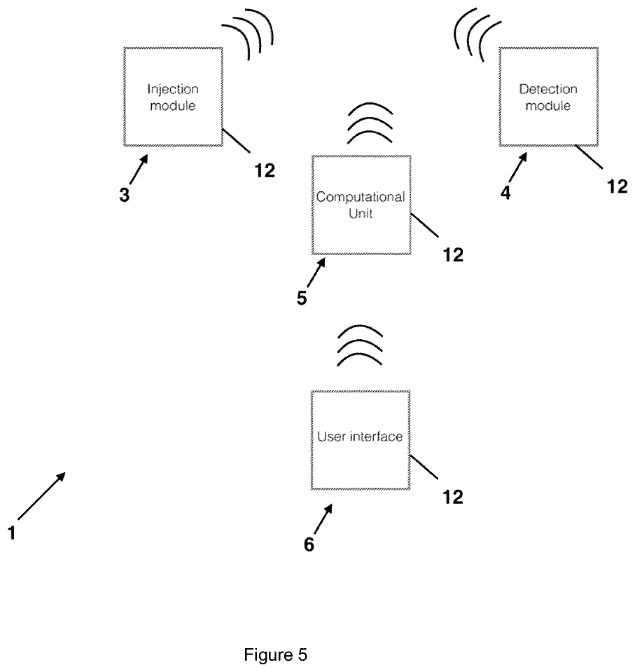

By comparing the system configurations and constructions illustrated in FIGS. 2, 3 and 5, the person skilled in the art will understand that the preferred four functional units 3, 4, 5, 6 of the system 1 may be either incorporated, partly or completely, in a more global medical system 10 for example an anesthesia apparatus (rectangle of broken lines of FIG. 2); all accommodated together in a single housing 11 (see FIG. 3) or each incorporated separately in its own independent housing 12 (see FIG. 5).

The three main constitutive modules 3, 4 and 5 will now be described in more details, according to various possible embodiments and in relation to different constructive alternatives of the system 1.

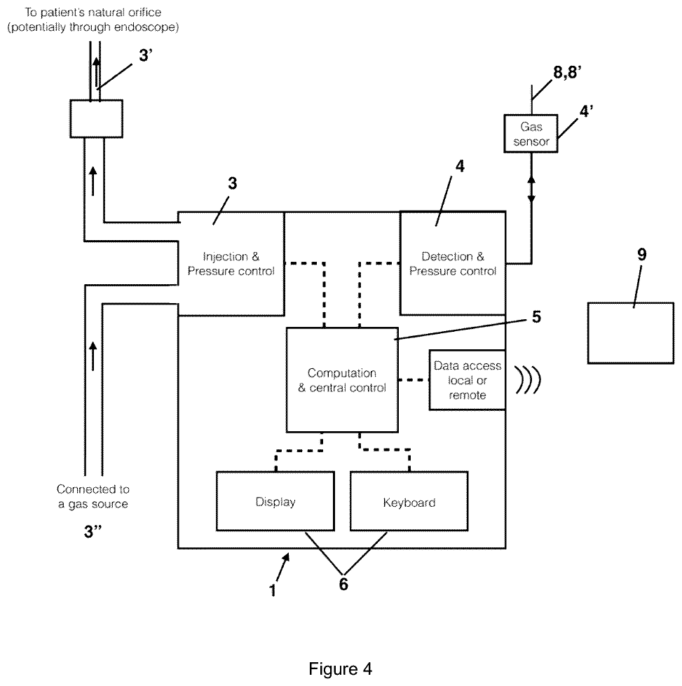

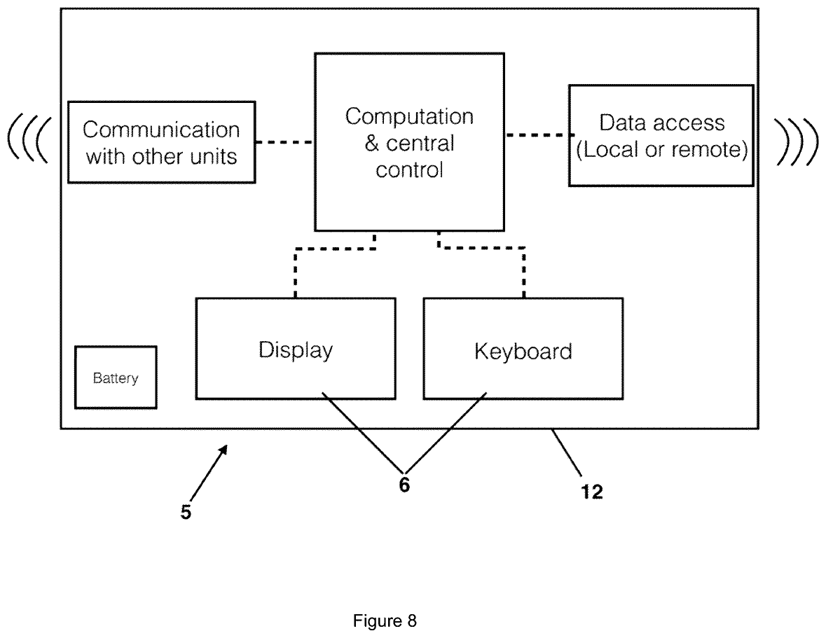

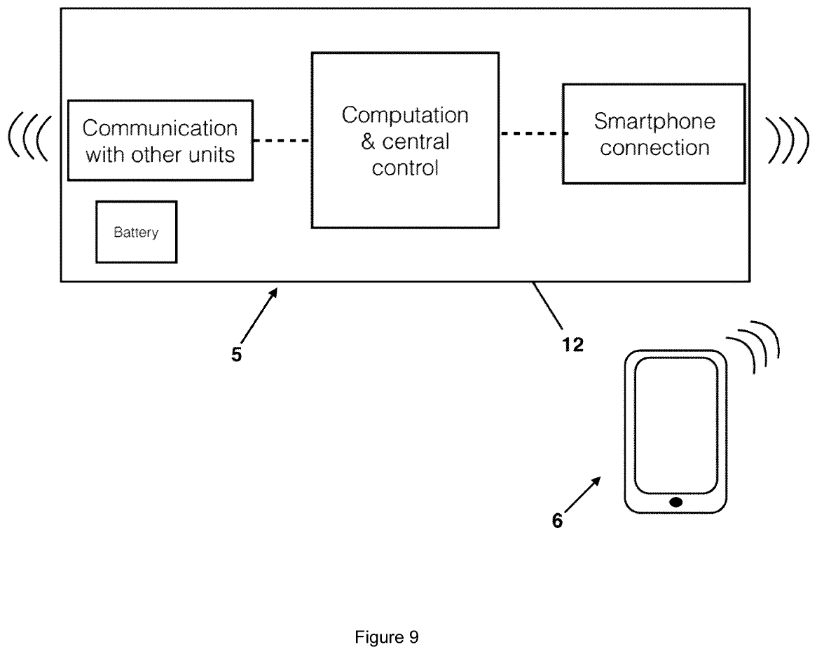

Computational Module (FIGS. 4, 8 and 9)

The computational module 5 acts as the central unit of the system 1. Based on stored data and information entered by the user, it computes parameters of the leak test to be performed and controls the detection module 3 and the injection module 4 to perform the leak test. During the leak test, the computational module 5 receives and interprets continuously information sent by the detection module 4 and the injection module 3.

Said computational module 5 has computational power, communication capabilities with the injection module 3, the detection module 4 and a possible user interface 6. Said computational module also comprises remote and/or local data storage 9.

The computational module 5 may send instructions to the injection module 3 and the detection module 4 and can receive information from them. The computational module 5 can send information to the user interface 6 to be displayed to the user and can interpret information coming from the user interface 6. The computational unit 5 can access and interpret data from both local and remote storages (local storage means may be integrated with the communication and computation means in hybrid circuits or as separate circuits on electronic PCB).

The computational module is adapted to interpret information entered by the user, which may include parameters describing the clinical setting and potentially influencing the measurement such as for example: weight, age, sex of the patient, site of potential perforation, indication about the estimated size of the perforation.

When the modules are mounted in separate housings 12, the computational module 5 ensures the communication with the detection module 4 and the injection module 3. It also displays the information that the system 1 is ready to perform a leak test to the user on the particular user interfaces 13′ integrated in the computational module 5, in the detection module 4 and in the injection module 3. The user then places and connects the cooperation means integrated in the detection module 4 and in the injection module 3, depending on the clinical setting and on the organ to be checked.

By using another of the specific user interfaces 13′ (input), the user can then start the leak test.

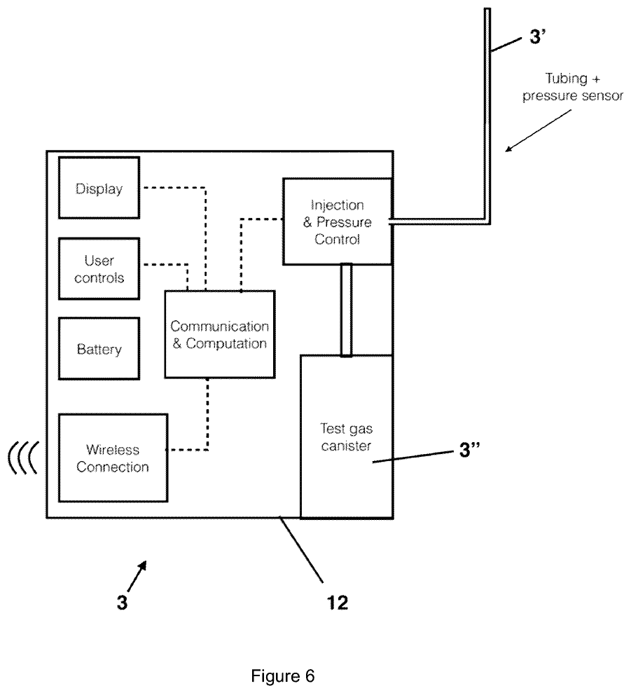

Injection Module (FIGS. 4 and 6)

The injection module 3 is intended to inject a test gas TG inside a hollow organ 12 to be tested and to control and measure parameters of this injection such as the volume of gas injected, the concentration and/or the rate of injection, and also the pressure at the injection site.

In a general embodiment, the injection module comprises a connection to a pressurised source 3″ of test gas TG, a gas carrying element, means to control the inlet of test gas in the gas carrying element 3′ and means to communicate with the computational unit 5 and to interpret information received.

In one embodiment, the pressurised source 3″ of test gas is a disposable container and the injection module comprises a connection system configured to adapt to the container. In another embodiment, the pressurised source of test gas may be a gas delivery line of a corresponding distribution network.

In one embodiment, the injection module 3 comprises a communication means to exchange information or instructions with the detection module 4 and/or the computational module 5 and means (computational resources) to interpret any information or instructions received. Information and instructions transferred include but are not limited to, beginning and end of the gas injection, volume of gas injected, type of gas injected, pressure at the injection site (see FIG. 6).

According to the present invention, the injection module 3 comprises a test gas TG carrying element 3′ to carry the test gas to the test site. The test gas can be carried to the desired site by different entries/approaches depending on the clinical setting.

In one embodiment, the gas carrying element is a tube 3′ adapted for insertion in the human body 2′. Such a gas carrying element can be manufactured in a bio-compatible material, for example a plastic or metal. For example, in the case of colorectal surgical procedures, the gas carrying element is adapted for anal insertion and may include means to seal the anal orifice.

In another embodiment, the gas carrying element is adapted to be inserted inside the working channel of a diagnostic or surgical apparatus such as a flexible endoscope (not shown).

In another general embodiment, the injection module 3 and the computational module 5 may not be integrated in the same housing. In such an embodiment, said communication means of said injection module 3 may for example be radio-frequency or wire based (FIG. 6).

In one embodiment, the injection module 3 may comprise an independent power supply such as batteries or means to connect it to a standard power outlet (FIG. 6).

In one embodiment, the injection module 3 comprises a specific user interface comprising means 13 to display information to the user such as, but not limited to, confirmation of the data connection between said injection module 3 and said computational module 5, beginning and ending of the injection of said test gas, the amount of gas remaining in the container 3″, an indication of the pressure level inside the gas container 3″, an indication of the general status of the injection module 3, an indication of the pressure at the injection site.

Said injection module 3 may also comprise a specific user interface 13′ with user controls, enabling a user to control the injection of said test gas and comprising for example controls such as: stopping, starting or controlling parameters of said injection of said test gas or turning the injection module on or off or settings designed for differing organ systems.

In one embodiment, the injection module 3 is integrated in an independent housing 12 and may be adapted to be used as a handheld instrument. In this embodiment, the housing may for example be adapted to be connected to a disposable canister 3″ filled with the test gas.

In another embodiment, the injection module 3 may be adapted to be used during a flexible endoscopy procedure. The injection module 3 may thus be substantially compact and may include a means to anchor the injection module to the handle of a flexible endoscope. The injection module may also be reversibly connected to the gas carrying element 3′.

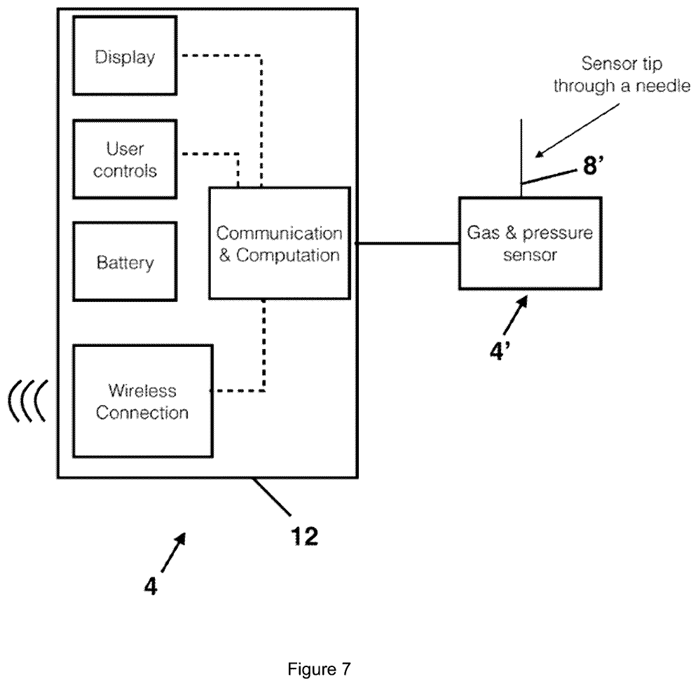

Detection Module (FIGS. 4 and 7)

The purpose of the detection module 4 is to make a real time measurement in order to determine the presence and/or concentration of a test gas in a region adjacent an organ 2 to be checked. The detection module 4 is adapted to measure parameters such as (but not limited to) the concentration of at least one specific gas, or the pressure at the measurement site.

In an embodiment, the detection module comprises a sensor 4′ adapted to detect the test gas TG, an access device 8 granting access to the zone adjacent to the hollow organ 2, means to communicate with the computational module 5 and means to interpret information received from the computational module (computational resources).

In one embodiment, the detection module 4 comprises means to communicate information to the injection module 3 and/or the computational module 5 and means to interpret any information or instructions received. Transferred information and instructions include but are not limited to, beginning and end of the gas measurement, concentration of gas measured, pressure at the measurement site.

According to the present invention, the detection module 4 also comprises an access element 8 to either introduce the measuring element (tip of the sensor 4′) within the body cavity 7 containing the organ 2 and preferably adjacent said organ 2 to be checked or, in cooperation with transfer tubing 8′, to carry the gas or gas mixture (GM) potentially present from a zone adjacent the organ 2 to be checked to the measuring element and analysing means of the detection module 5 located outside the body. The access element 8 can vary depending on the clinical setting.

In a general embodiment, the access element 8 comprises a needle to be inserted through a biological tissue to reach a zone adjacent to the organ 2 to be checked.

In one embodiment, the measurement means 4′ is placed within the passageway of the hollow shaft of the needle 8 and introduced in said cavity 7 through said passageway.

In another embodiment, gas is carried through the shaft of the needle 8 to the measurement means 4′, for example by using a hollow connector and tubing 8′.

In another general embodiment, in the case of laparoscopic surgery, the access element 8, for example a hollow tube, may be connected to a trocar. This configuration allows the system to be used quickly.

In one embodiment, the detection module 4 may comprise an independent power supply such as batteries or means to connect it to a standard power outlet.

In another embodiment, the detection module 4 comprises a specific user interface comprising means 13 to display information to the user such as, but not limited to, confirmation of the data connection between said detection module 4 and said computational module 5, beginning and ending of the detection of said test gas, a real time indication of the concentration of test gas or fluid measured, an indication of the general status of the detection module, an indication of the result of the computation, that is whether the system has determined that a leak was present or not. Said user interface of said detection module 4 may also comprise user controls 13′ enabling a user to control the detection of said test gas comprising for example controls such as stopping or starting said detection of said test gas.

In one embodiment, the detection module is integrated in an independent housing and may be adapted to be used as a handheld instrument.

The following concerns a handheld leakage detection device 201, for use in a surgery.

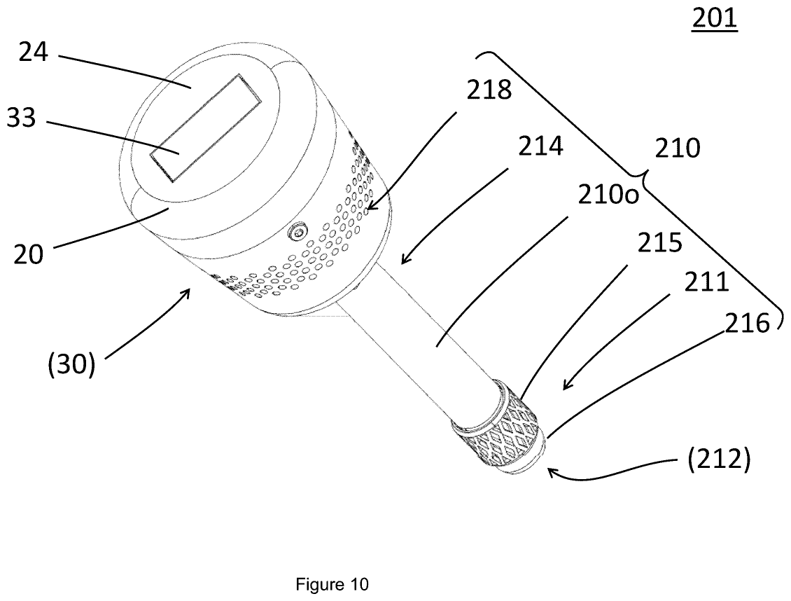

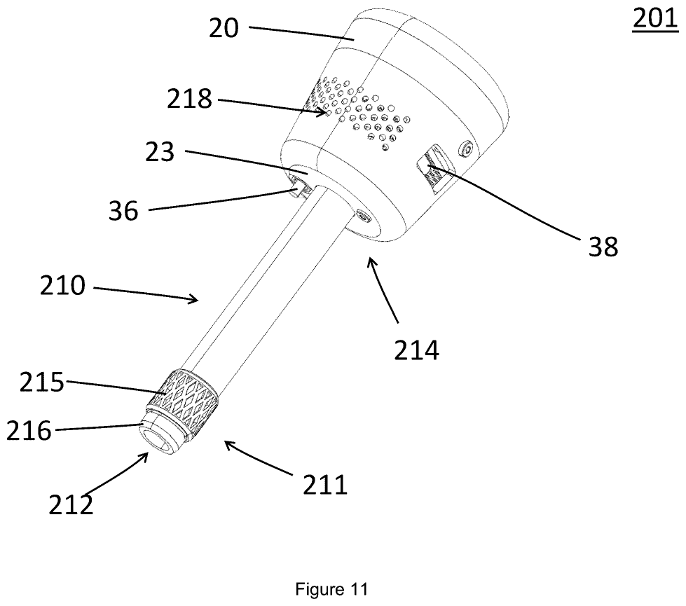

FIGS. 10 and 11 illustrate a preferred embodiment of the detection device 201, intended to cooperate with a trocar 100. It mainly comprises:
an access element 210 intended to be in connection with the body cavity 7 wherein an organ 2, comprising an anatomical conduit, is under surgery,
a housing 20 connected to the access element 210,
a detection module 30.

The detection device 201 features an access element 210 extending along an axis, and comprising a proximal end 214 on the side of the housing 20, and a distal end 211 on a free side of the access element 210. The distal end 211 is configured to be in connection with the body cavity 7.

The access element 210 is preferably in the shape of a hollow cylinder revolving around the axis, traversed by a lumen 212, and extending from the distal end 211 in the direction to the proximal end 214, thus to the housing 20.

A gas sensor 31 is disposed within the lumen 212, thus allowing a gas mixture GM contained by the body cavity 7 to be in contact with the gas sensor 31. Said gas sensor 31 can then detect in the gas mixture GM the presence or an abnormal quantity of a test gas TG insufflated in the hollow organ 2, and which can have leaked from the insufflated hollow organ 2. The gas sensor 31 can also detect the pressure inside the body cavity 7.

In order to ensure the holding of the detection device 201, the access element 210 comprises an adjustment member 215, intended to be radially tight against the internal surface 111 of the cannula 110 of the trocar 100. To that end, the adjustment member 215 can be made of an elastomeric material. The adjustment member 215 can feature an external diameter slightly superior than an inner diameter of the internal surface 111.

The adjustment member 215 can be in the shape of a bush, placed in a circumferential recess of an outer surface 210o of the access member 210. The adjustment member 215 can be of any other suitable shape, e.g., protrusions from the outer surface 210o and so on.

The adjustment member 215 can further have a sealing function against the internal surface 111, preventing a leakage of the gas mixture GM through a radial space between the outer surface 210o of the access element 210, and the internal surface 111 of the cannula 110.

In this case, an elastomeric material is preferred for the adjustment member 215. A bush shape is also preferred.

The proximal end 214 of the access element 210 is preferably mounted directly on the housing 20 which contains a sub-computational module 32 of the detection device 201. This rigid and compact assembly allows the detection device 201 to be handled by a single hand.

An alternative not shown resides in a tubing connection through the access element 210 and the housing 20, with a flexible pipe. In order the detection device 201 remains handheld, the length of the pipe should not excess a few centimeters, e.g., 10 cm or 20 cm.

The housing 20 contains the sub-computational module 32, for receiving the gas parameter acquired by the gas sensor 31, and process it. A user interface 33 is placed on the top 24 of the housing 20 to display the result of the analysis conducted by the sub-computational module 32.

The user interface 33 can be a screen displaying an information. Means for the surgeon to prompt information for the sub-computational module 32 may not be needed.

The user interface 33 can be a color-changing LED, the color of the LED indicating the potentiality of the presence of a leakage L, according to a color-based code.

The user interface 33 can be a sound emitting module, configured to emit a sound alarm if the probability of a leakage L is above a predefined threshold.

The user interface 33 can be a wireless connection, as a Bluetooth connection, configured to display the information on an electronic device paired with the detection device 201, as a smartphone.

Preferably the user interface 33 is a screen, e.g., displaying the concentration of a specific gas among the gas mixture GM. It can be the concentration of the test gas TG, which will raise in case of a leakage L. It can be the concentration of carbon dioxide, used to insufflate the body cavity 7.

If a leakage L occurs, the test gas TG will replace some carbon dioxide among the gas mixture GM. Thus, the concentration of carbon dioxide will decrease in case of a leakage L.

The user interface 33 is preferably placed on a top side 24 of the housing 20, opposite to the access element 210, in order to be clearly visible from the surgeon point of view when the detection device 201 is mounted into the trocar 100.

In reference to FIG. 11, a switch 36 is placed on an under-face 23 of the housing 20. This allows the switch 36 to be activated by a top surface 102 of the trocar 100 head when the detection device 201 is mounted into the trocar 100. This switch 36 is used to turn the detection device 201 on and off: the detection device 201 according to this feature is by consequence a “plug and play” detection device 201, which automatically turns on when it is mounted into the trocar 100, and automatically turns off when the detection device 201 is dismounted from the trocar 100.

A such plug and play detection device 201 allows a very quick and easy installation and deinstallation during the surgery. It is thus easy to obtain information as to the presence of a leakage L, on a regular basis, during the surgery.

This feature also helps to save the energy of the power supply 34 included in the housing 20.

The switch 36 can be placed elsewhere on the detection device 201, e.g., on the outer surface of the access element 210. In that case, the switch 36 can be activated by the mating inner surface 111 of the cannula 110 of the trocar 100.

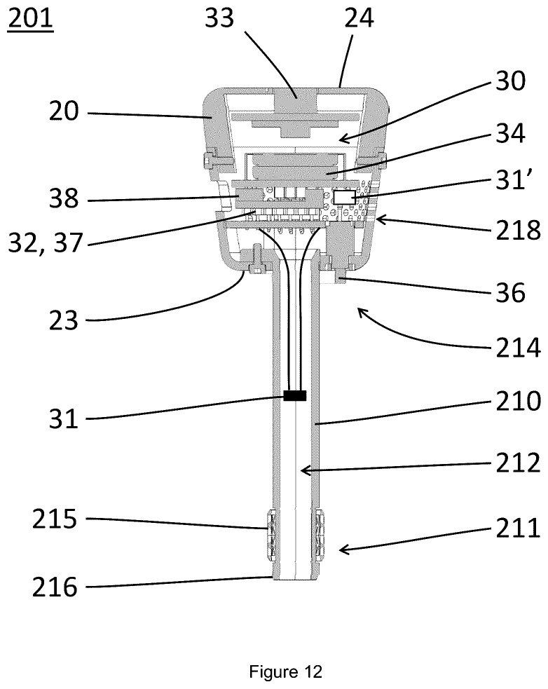

Referring to FIG. 12, the housing 20 includes:
the sub-computational module 32, which receives the sensing parameters and compute them to display a leakage information LI,
the user interface 33,
and the power supply 34, for powering the sub-computational module 32, the sensors 31, 31′, and so on.

Preferably, the housing 20 further includes a data storage device 37, and a data transfer interface 38. The latter is preferably a wire-type connector, in order to avoid the need to pair the detection device 201 with a computer, intended to receive the stored data after the surgery.

The housing 20 may further comprise a supplementary sensor 31′, intended to measure a supplementary gas parameter, as the pressure of the test gas TG insufflated in the hollow organ 2.

In that case, the detection device 201 features a fluidic connection with the insufflator 200 from the injection module 3, in order to link the supplementary sensor 31′ with the insufflated test gas TG. This connection can be easily made by adding a T shaped connector to the tube linking the insufflator 200 to the body part 14, and directing a supplementary tube, connected to the T-shaped connector, to a connector of the housing 20.

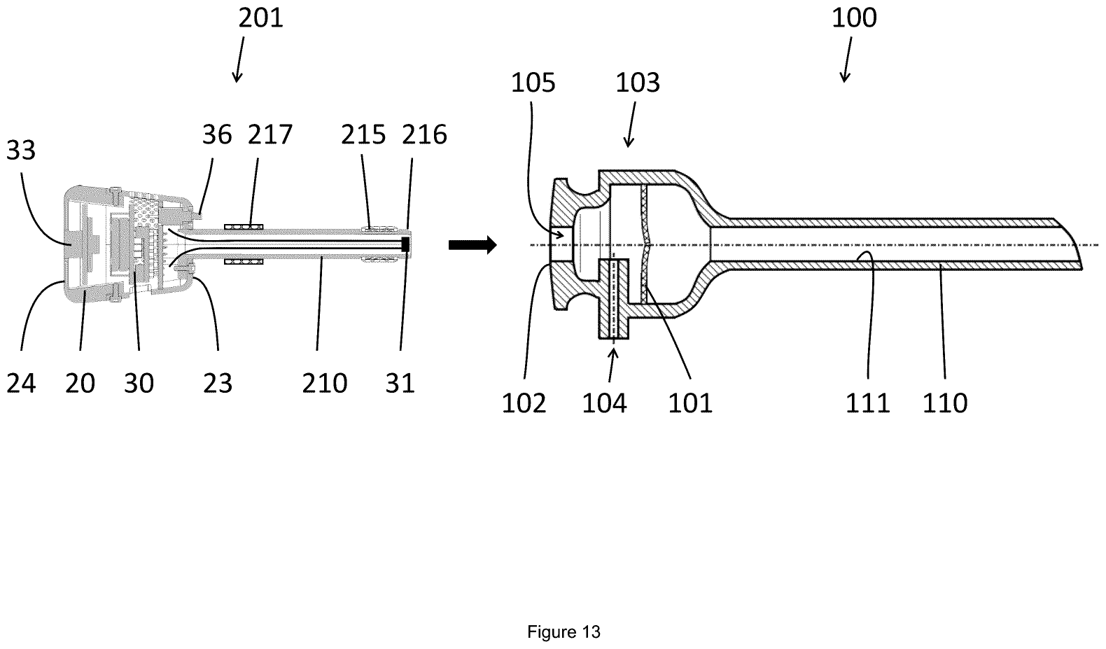

FIG. 13 illustrates the detection device 201 and the trocar 100, before their assembly.

The trocar 100 comprises a cannula 110 configured to penetrate the body cavity 7 through the skin 15, and a trocar 100 head which features:
an access hole 105 designed to allow surgical instruments to access the body cavity 7 through the cannula 110,
a sealing valve 101 preventing the leakage L of the gas mixture GM insufflated in the body cavity 7 to provide the needed space during the surgery,
eventually, a connection hole 104 which can be used to insufflate the gas mixture GM into the body cavity 7. The presence of the connection hole 104 relies on the type of the trocar 100.

The access element 210 preferably comprises a passing member 216 configured to spread and pass the sealing valve 101 without damaging it. The passing member 216 can be a rounded shape, or a chamfer located at the distal end 211.

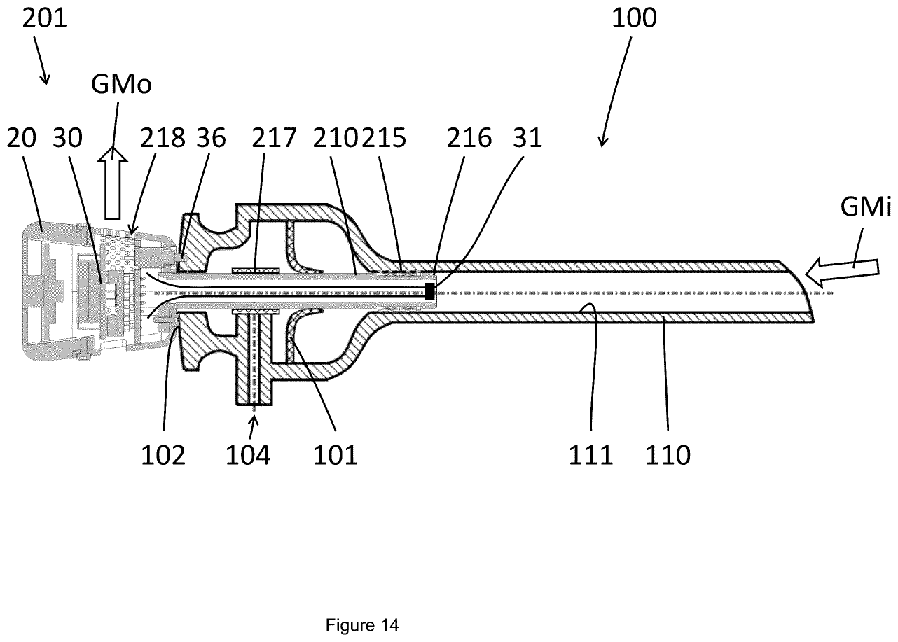

The FIG. 14 illustrates the detection device 201 mounted into the trocar 100.

The access element 210 has passed through the sealing valve 101 and has reached the cannula 110.

The adjustment member 215 is radially pressing against the inner surface 111 of the cannula 110, thus preventing an unwanted dismounting of the detection device 201 and trocar 100.

Preferably, the sealing valve 101 is radially tight with the access element 210, in order to prevent unwanted leakage L of the gas mixture GM. Moreover, this tightness contributes to the correct holding of the detection device 201.

A sealing member 217 is mating with the connection hole 104 and seals it, also to prevent the leakage L of the gas mixture GM.

An upper surface 102 of the trocar head 103 has pushed the switch 36, automatically turning on the detection device 201, therefore starting the leakage analysis.

The housing 20 comprises a gas outlet 218 to generate a flow of the gas mixture GM from the body cavity 7 to the distal end 211, through the lumen 212, and then to the gas outlet 218.

The gas mixture GM flows from a gas inlet GMi at the distal end 211 to the gas outlet 218 where it exits the detection device GMo. The gas sensor 31 being placed inside the lumen 212, it is surrounded by the gas mixture GM from the body cavity 7.

This flow is generated by the slight overpressure residing inside the body cavity 7 during this kind of surgery.

This flow further helps the potentially leaking test gas TG to leave the surroundings of the leakage L, and to ensure the leaking test gas TG to reaches the gas sensor 31.

The gas outlet 218 can be adapted in size and geometry in order to adapt the flowrate of the gas mixture GM leaving the body cavity 7.

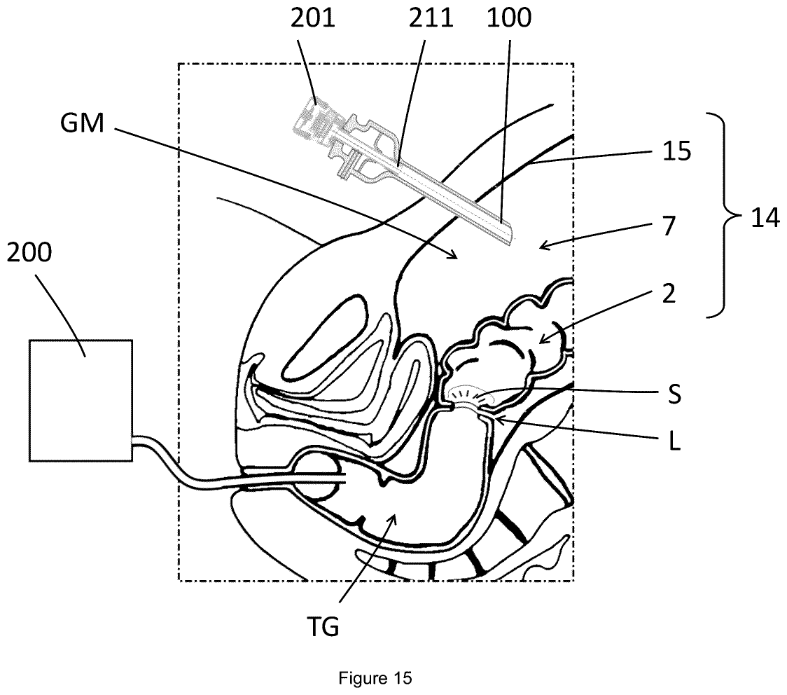

FIG. 15 illustrates a leakage system being used during a surgery. At certain point during the surgery, the surgeon may want to verify if there is a leakage L on the organ 2 he is working on. He can remove the surgery instruments form the trocar 100, place instead the detection device 201 for the duration of the detection.

An insufflator 200 fills the hollow organ 2 with a test gas TG, in order to verify if the suture S made by the surgeon presents a leakage L.

If so, some test gas TG will reach the body cavity 7 and be present in the gas mixture GM herein.

The distal end 211 of the detection device 201 being placed inside the cannula 110 of the trocar 100, the sensor not shown on FIG. 6 can acquire the needed test gas parameter TGP.

According to the results, he can take appropriate measures, i.e., either carry on the surgery if no leakage L is occurring, or treat the organ 2 if a leakage L has been detected.

The surgeon can remove the detection device 201 and place back the surgery instruments instead. Using the same trocar 100 for these two purposes allows to avoid a further incision in the body to place the detection device 201. This method is preferred when a punctual surveillance is sufficient. In alternative, when a continuous surveillance is required, the detection device 201 can be mounted on a dedicated trocar 100. The surveillance is more thorough, but the drawback is the need to make a supplementary incision in the body.

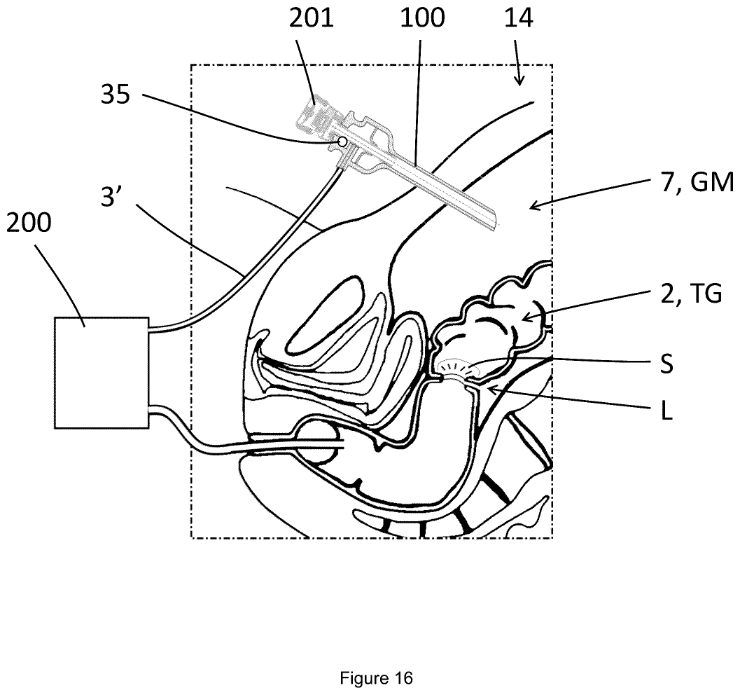

FIG. 16 illustrates an embodiment wherein the housing 20 comprises a supplementary sensor 31′ configured to measure a supplementary parameter, as the nature or the pressure of the insufflated test gas TG delivered to the detection device 201 through an carrying element 3′.

In the represented embodiment of FIGS. 15 and 16, although not limited thereto, the detection device 201 is implemented during a surgery, in particular a laparoscopy. The detection device 201 can however find other implementations in other surgeries or any other suitable purpose such as a follow-up further to a surgery.

In that case, the sub-computational module 32 can proceed to further methods for leakage L detection, for example base on differential pressure as described in document EP3838119, in particular pages 5 to 6.

The supplementary sensor 31′ can also be a temperature sensor, or any sensor needed.

Another embodiment allowing the use of a supplementary parameter reside in a wireless connection between the detection device 201 and the insufflator 200. In that case, the tube illustrated on FIG. 16 is no longer necessary, which is more ergonomic during the surgery. Nevertheless, it requires pairing the detection device 201 with the insufflator 200, so as the sub-computational module 32 can communicate with the insufflator 200, and receive an external parameter from an external sensor of the insufflator 200.

The illustrated access element 210 is hollow and the gas sensor 31 is placed inside the lumen 212. The access element can also be a solid shaft. In that case, the gas sensor 31 can also be put on an outer surface of the access element 210.

In either way, once the detection device 201 is mounted on the trocar 100, the gas sensor 31 is in contact with the gas mixture GM and operable for the gas detection.

For easing the understanding of the invention, only the cooperation with a trocar 100 has been illustrated, but it is to be understood that the invention relies on the handheld and the autonomous features of the detection device 201, which can be of any form or any conception.

The detection device 201 can be in the shape of an all-in-one leakage detecting trocar, or a Veress needle, wherein the access element 210 is configured to penetrate the body cavity 7 by cutting the skin 15.

The disclosed minimally-invasive method and system for assessing the integrity of an organ or lumen may provide numerous advantages. For instance, they may enable the objective detection of an ongoing gas leak, confirming that further endoscopic or operative management is required.

Additionally, they may provide objective evidence of adequate repair, which may avoid performance of a negative diagnostic laparoscopy or prevent invasive procedures such as a colostomy to be performed. Moreover, they may prevent invasive procedures in a large portion of patients, as the Veress other needle or a trocar may have already been placed intraoperatively.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments illustrated and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

1. A detection device for use in providing a gas information from a gas mixture contained within a cavity of a body part of a living being, and the body part comprising a skin layer, delimiting the cavity, the detection device comprising:
an access element presenting a distal end and configured to be in communication with the cavity,
a housing connected to the access element,
a detection module,
wherein the access element and the housing are configured to be held by hand of a user, and the detection module comprises:
at least one sensor comprising a gas sensor arranged on the access element and configured to measure at least one gas mixture parameter related to the gas mixture within the cavity,
a sub-computational module arranged in the housing and connected to said at least one sensor, the sub-computational module being configured to compute the gas mixture parameter to provide the gas information,
a user interface arranged in the housing and connected to the sub-computational module, the user interface configured to communicate gas information,
a power supply arranged in the housing and configured to supply the detection module with energy.

2. The detection device according to claim 1, wherein the sub-computational module is configured to communicate and receive an external parameter from an external sensor, and the sub-computational module is configured to compute the external parameter along with the gas mixture parameter.

3. The detection device according to claim 1, wherein it comprises a supplementary sensor configured to measure at least one supplementary parameter, and the sub-computational module is configured to compute the supplementary parameter along with the gas mixture parameter.

4. The detection device according to claim 1, wherein the access element comprises a gas outlet at a proximal end opposite the distal end.

5. The detection device according to claim 1, wherein it comprises a gas connection device configured to:
communicate with a test gas insufflator, and
receive a test gas parameter, and
the sub-computational module is configured to compute the test gas parameter along with the gas mixture parameter.

6. The detection device according to claim 1, wherein the access element presents a proximal end opposite the distal end, the housing being attached to the proximal end of the access element.

7. The detection device according to claim 1, wherein the housing comprises a data storage, configured to store gas parameter data from the sensor, or configured to store gas information.

8. The detection device according to claim 1, wherein the gas information is representative of a leakage in an anatomical conduit within the cavity.

9. The detection device according to claim 1, for use with a trocar having a cannula defining a passage to a free end and intended to be inserted in the body part through the skin layer, wherein the access element is configured to penetrate the cannula with the gas sensor in fluid communication with the passage, so that the gas sensor is in contact with the gas mixture when the cannula is arranged within the cavity.

10. The detection device according to claim 9, wherein an adjustment member disposed on an outer surface of the access element is configured to be radially tight against an internal surface of the cannula.

11. The detection device according to claim 9, wherein the access element is configured to be radially tight against a sealing valve.

12. The detection device according to claim 9, wherein a switch controlling an activation and a deactivation of the detection module is arranged on an external surface of the detection module, and is configured to be actuated by the trocar when the detection module is mounted on the trocar.

13. The detection device according to claim 9, wherein a sealing member on an outer surface of the access element is configured to obturate a connection hole of the trocar configured to insufflate the gas mixture into the body cavity, when the access element penetrates the cannula.

14. A system comprising:
a trocar, comprising a cannula which is intended to be inserted, through a skin layer delimiting a cavity of a body part of a living being, so that the cannula is arranged within the cavity which is filled by a gas mixture; and
a handheld detection device according to claim 9, and configured to be inserted in the cannula so that the distal end is in communication with the cavity, and so that the sensor measures at least one gas mixture parameter related to the gas mixture.

15. A system comprising:
an insufflator intended to inject a test gas into an anatomical conduit within the cavity; and
a handheld detection device according to claim 5, so that the sensor measures at least one gas mixture parameter related to the test gas.
